(19) United States
(12) Patent Application Publication
(10) Pub. No.: US 2011/0206763 A1
(43) Pub. Date: Aug. 25, 2011

(54) RAPID-RELEASE ENCAPSULATION COMPOSITION

(75) Inventors: Tom Keenan, Sioux City, IA (US); John M. Dolphin, Sioux City, IA (US)

(73) Assignee: Gelita, Eberbach (DE)

(21) Appl. No.: 13/030,679

(22) Filed: Feb. 18, 2011

Related U.S. Application Data
(60) Provisional application No. 61/306,744, filed on Feb. 22, 2010.

(51) Int. Cl. A61K 9/64; A61K 47/42; A61K 47/02; A61P 1/04

(52) U.S. Cl. 424/456; 514/774; 514/769

(57) ABSTRACT

A rapid-release encapsulation composition that includes a gelatin and a water-insoluble rapid-release agent is provided. In particular, a rapid-release encapsulation composition that includes a gelatin and an insoluble carbonate salt is provided.

### CROSS-REFERENCE

This application claims priority to U.S. provisional application Ser. No. 61/306,744, filed Feb. 22, 2010, the contents of which are hereby incorporated by reference in its entirety.

### FIELD OF THE INVENTION

The current invention relates to rapid-release encapsulation compositions. More specifically, the invention relates to rapid-release encapsulation compositions that include a gelatin component and a rapid-release agent.

### BACKGROUND OF THE INVENTION

The development of compositions for the rapid release of active pharmaceutical compounds (APIs) is an ongoing challenge in the pharmaceutical industry. Due to various unpalatable characteristics of many APIs such as bitterness, some oral compositions incorporate added flavorants to mask the unpalatable flavor of the API. Further, other oral pharmaceutical compositions include an encapsulant to isolate the API during oral administration, to alleviate other unpalatable characteristics of the API such as grittiness or stickiness, and to enhance other properties of the composition such as stability during storage and/or transport. Encapsulated pharmaceutical compositions such as gelatin-coated tablets, hard capsules, and soft gelatin capsules are widely used for orally administered therapeutic compositions.

Although the encapsulation of APIs alleviates many of the palatability and stability issues described above, the encapsulation material properties pose a further challenge with respect to the rapid release of the API into the gastric cavity. Some existing approaches make use of a water-soluble encapsulant material, but many water-soluble encapsulants tend to partially dissolve in the oral cavity or esophagus causing sticking during oral administration. Other approaches make use of pH-sensitive coatings that incorporate polymeric materials that are relatively insoluble in a relatively neutral pH environment such as the oral cavity, but are highly soluble in an acidic environment such as the gastric cavity; however, the production of pH-sensitive polymer encapsulants typically requires specialized production techniques.

Other existing rapid-release compositions incorporate various water-soluble porigenic materials into the encapsulation material that dissolve in the gastric cavity and form pores in the remaining encapsulation material, allowing the API within to dissolve and diffuse outward into the gastric cavity. Yet other existing rapid-release compositions include a multilayer encapsulant in which a waterproof porous outer layer conducts gastric juices to a swellable inner layer material, which bursts the encapsulant upon swelling and exposes the underlying API. These encapsulant compositions require considerable effort to manufacture and require the penetration of the gastric juices through pores in an outer coating layer to implement the release of the API, resulting in delayed release times for the APIs in the gastric cavity.

Gelatin is a well-established encapsulation material in the pharmaceutical industry. The material properties of gelatin may be adjusted or controlled by gelatin treatment methods such as cross-linking, deionization and partial hydrolysis to produce gelatin coatings with specified material properties such as rigidity and solubility. The production of gelatin coatings, soft gelatin capsules, and hard gelatin capsules typically utilizes an aqueous suspension of the gelatin. As a result, it is difficult to incorporate water-soluble rapid-release additives to enhance the rapid-release properties of the resulting gelatin encapsulant in the gastric cavity. Further, gelatin rapid-release additives may alter the chemical properties of the gelatin suspension such as pH, which may cause degradation or instability of the resulting gelatin matrix structure.

A need exists in the art for a gelatin-based rapid-release encapsulation composition that rapidly degrades in the acidic environment of the gastric cavity, but not in pH-neutral aqueous environment of the oral cavity and esophagus during oral administration of the API. Further, a need in the art exists for rapid-release additives to enhance the rapid-release properties of gelatin or other polymer encapsulants in the gastric cavity that do not degrade the encapsulant's matrix structure during production. Such a rapid-release additive would facilitate the production of rapid-release encapsulations using well-established encapsulation technologies.

### SUMMARY OF INVENTION

Among the various aspects of the invention, therefore, is the provision of a rapid-release encapsulation composition that includes a water-insoluble rapid-release agent and a gelatin. It has been discovered that the rapid release agent dissociates and releases gas bubbles in the acidic environment of the gastric cavity causing the rapid degradation of the gelatin due to the bubbles physically tearing at the gelatin during release. As a result, the use of the rapid-release encapsulation composition as a dosage form for an ingestible product such as an oral therapeutic compound results in the rapid release of the compound in the gastric cavity.

The rapid-release encapsulation composition may further include a gelatin hydrolysate. Another aspect provides a rapid-release encapsulation composition that includes calcium carbonate and a gelatin, in which the composition has a mass ratio of the calcium carbonate to the gelatin ranging from about 1:1 to about 1:20. The mass ratio of two compounds in a composition, as used herein, refers to the numerical quantity resulting from dividing the mass of the first compound contained in the composition by the mass of the second compound contained in the composition. Yet another aspect provides a rapid-release encapsulation composition that includes a gelatin dissolved in an aqueous solution as well as a water-insoluble rapid-release agent suspended in the aqueous solution. Still another aspect provides a rapid-release encapsulation composition that includes calcium carbonate, a gelatin, and a gelatin hydrolysate, in which the gelatin hydrolysate has a molecular weight in the range from about 100 Daltons to about 2000 Daltons.

The rapid-release agent is selected to be relatively insoluble in aqueous solutions having a pH ranging from about 6 to about 8. This property of the rapid release agent prevents the rapid-release agent from dissolving and reacting with other ingredients during the production of the rapid-release encapsulation composition, which typically makes use of an aqueous solution of the gelatin and the rapid-release agent. In addition, the rapid-release agent is selected to dissociate rapidly in an aqueous solution having a pH ranging from 0 to about 3. In use as an encapsulant of an orally administered therapeutic composition, the rapid-release agent remains relatively inert in the buccal cavity, in which the pH is typically from about 6 to about 7. Upon contacting the gastric juices within the gastric cavity, in which the pH typically ranges from about 1 to about 3, the rapid-release agent dissociates rapidly, resulting in the rapid release of the active pharmaceutical ingredient of the therapeutic composition.

Still another aspect provides a rapid-release hard capsule shell composition that includes a gelatin and a water-insoluble rapid-release agent, in which the composition has a mass ratio of the water-insoluble rapid-release agent to the gelatin ranging from about 1:1 to about 1:20. An additional aspect provides a rapid-release soft-gel capsule encapsulation composition that includes a gelatin and a water-insoluble rapid-release agent in which the composition has a mass ratio of the water-insoluble rapid-release agent to the gelatin ranging from about 1:1 to about 1:20. In another additional aspect, a rapid-release tablet coating composition that includes a gelatin and a water-insoluble rapid-release agent in which the composition has a mass ratio of the water-insoluble rapid-release agent to the gelatin ranging from about 1:1 to about 1:20 is provided. Any of the above rapid-release encapsulation compositions may further include a gelatin hydrolysate.

A chewable therapeutic composition is provided in another aspect that includes a plurality of active pharmaceutical ingredient particles encapsulated in a rapid-release coating. In this aspect, the rapid-release coating includes a gelatin and a water-insoluble rapid-release agent, and the composition has a mass ratio of the water-insoluble rapid-release agent to the gelatin ranging from about 1:1 to about 1:20. The rapid-release coating may further include a gelatin hydrolysate.

A therapeutic chewing gum composition is provided in yet another aspect that includes a plurality of active pharmaceutical ingredient particles encapsulated in a rapid-release coating in which the rapid-release coating includes a gelatin and a water-insoluble rapid-release agent. The composition in this aspect has a mass ratio of the water-insoluble rapid-release agent to the gelatin ranging from about 1:1 to about 1:20. The rapid-release coating may further include a gelatin hydrolysate.

A further aspect of the current invention provides a method for manufacturing a rapid-release encapsulation composition comprising the steps of: (a) dissolving a gelatin component in an aqueous medium; and (b) adding a water-insoluble rapid-release agent to the aqueous gelatin solution. The gelatin component of step (a) may include a combination of a gelatin having a molecular weight ranging from about 50,000 Daltons to about 300,000 Daltons and a gelatin hydrolysate having a molecular weight ranging from about 100 Daltons to about 2000 Daltons. The mass ratio of the gelatin having a molecular weight ranging from about 50,000 Daltons to about 300,000 Daltons to the gelatin hydrolysate having a molecular weight ranging from about 100 Daltons to about 2000 Daltons may range from about 3:1 to about 99:1, from about 4:1 to about 19:1, and from about 5:1 to about 13:1. The water-insoluble rapid-release agent of step (b) may include bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof. The aqueous medium of step (b) may include water. Generally, the rapid-release agent is essentially insoluble at a pH ranging from about 6 to about 8, and wherein the rapid-release agent dissociates at a pH ranging from 0 to about 3. Typically, the mass ratio of the water-insoluble rapid-release agent to the gelatin component ranges from about 1:1 to about 1:20, from about 1:2 to about 1:15, and from about 1:4 to about 1:9.

The method of manufacturing a rapid-release encapsulation composition may further include the addition of a plasticizer. Typical plasticizers include dibutyl sebacate, diethyl phthalate, glycerine, polyethylene glycol, propylene glycol, sorbitol, erythritol, triacetin, and triethyl citrate, and mixtures thereof. Generally, the gelatin component may include a combination of a gelatin, a plasticizer, and water, or a combination of a gelatin, a gelatin hydrolysate, a plasticizer, and water. In one embodiment of the method, step (a) may include dissolving about 0.01% to about 30% of the gelatin component by weight of the combined aqueous gelatin solution in about 40% to about 99.9% of the aqueous medium by weight of the combined aqueous gelatin solution. In another embodiment of the method, step (a) may include dissolving about 10% to about 20% of the gelatin component by weight of the combined aqueous gelatin solution in about 70% to about 90% of the aqueous medium by weight of the combined aqueous gelatin solution. In a further embodiment, step (a) may include dissolving about 10% to about 20% by weight of the gelatin having a molecular weight ranging from about 50,000 Daltons to about 300,000 Daltons and about 1% to about 5% by weight of the gelatin hydrolysate having a molecular weight ranging from about 100 Daltons to about 2000 Daltons in about 70% to about 90% aqueous medium by weight of the combined aqueous gelatin solution.

In yet another embodiment of the method of the current invention, step (a) comprises dissolving about 35% to about 60% gelatin by weight of the gelatin component, about 15% to about 30% plasticizer by weight of the gelatin component, and about 25% to about 40% water by weight of the gelatin component. Step (a) may also comprise dissolving about 42% to about 48% gelatin by weight of the gelatin component, about 20% to about 25% plasticizer by weight of the gelatin component, and about 30% to about 35% water by weight of the gelatin component.

In still another embodiment of the methods of the current invention, step (a) may comprise dissolving from about 32% to about 40% by weight of a gelatin having a molecular weight ranging from about 50,000 Daltons to about 300,000 Daltons, from about 17% to about 22% by weight plasticizer, from about 26% to about 31% by weight of water, and from about 2% to about 6% by weight of a gelatin hydrolysate having a molecular weight ranging from about 100 Daltons to about 2000 Daltons, and wherein step (b) comprises adding about 10% to about 15% by weight calcium carbonate.

Other aspects and iterations of the invention are described in detail below.

### DESCRIPTION OF FIGURES

The following figures illustrate various aspects of the invention:

FIG. 1 is a graph showing the measured dissolution in a pH=1 buffer solution of gelatin compositions that included various acid compounds.

FIG. 2 is a graph showing the effect of adding carbonate on the measured dissolution of a gelatin composition in a pH=1 buffer solution.

FIG. 3 is a graph showing the effect of storing a gelatin composition on its measured dissolution characteristics in a pH=1 buffer solution.

FIG. 4 is a graph showing the effect of storing a gelatin composition that includes calcium carbonate on its measured dissolution characteristics in a pH=1 buffer solution.

FIG. 5 is a graph showing the effect of storing a gelatin composition that includes calcium carbonate and gelatin hydrolysates on its measured dissolution characteristics in a pH=1 buffer solution.

FIG. 6 is a graph comparing the measured dissolution of three different gelatin compositions in a pH=1 buffer solution after 11 weeks of storage.

FIG. 7 is a graph showing the effect of storing a gelatin composition that includes calcium carbonate on its measured dissolution characteristics in deionized water.

FIG. 8 is a graph showing the effect of storing a gelatin composition that includes calcium carbonate on its measured dissolution characteristics in deionized water.

FIG. 9 is a graph showing the effect of storing a gelatin composition that includes calcium carbonate and gelatin hydrolysates on its measured dissolution characteristics in deionized water.

FIG. 10 is a graph comparing the measured dissolution of three different gelatin compositions in deionized water after 11 weeks of storage.

FIG. 11 illustrates the dissolution profile of the three gelatin formulations in simulated gastric fluid (at a pH of approximately 1.3, and in the absence of any enzymes). The three gelatin formulations include a bone gelatin without any further modifications (“Std Bone Gelatine”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 12 illustrates the dissolution profile of three gelatin formulations in water (approximately neutral pH levels) after the formulations were stored at 40° C. and 75% relative humidity for a period of two weeks. The three gelatin formulations include a bone gelatin without any further modifications (“Std Bone Gelatine”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 13 illustrates the dissolution profile of three gelatin formulations in simulated gastric fluid (at a pH of approximately 1.3, and in the absence of any enzymes), after the formulations were stored at 40° C. and 75% relative humidity for a period of two weeks. The three gelatin formulations include a bone gelatin without any further modifications (“Std Bone Gelatine”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

FIG. 14. illustrates the dissolution profile of three gelatin formulations in simulated gastric fluid (at a pH of approximately 1.3, and in the absence of any enzymes), after the formulations were stored at 40° C. and 75% relative humidity for a period of four weeks. The three gelatin formulations include a bone gelatin without any further modifications (“Std Bone Gelatine”); a bone gelatin as well as 15% calcium carbonate (CaCO₃) by weight (“RR only”); and a bone gelatin, 15% calcium carbonate (CaCO₃) by weight, and 10% by weight of hydrolyzed bone gelatin having a molecular weight of about 500 Daltons (“RR RXL”).

### DETAILED DESCRIPTION

### I. Composition

The invention includes rapid-release encapsulation compositions that include a water-insoluble rapid-release agent, such as a carbonate, and a gelatin component. The water-insoluble rapid-release agent is selected such that the rapid-release agent remains relatively insoluble in an aqueous solution ranging from a pH of about 6 to about 8, allowing the rapid-release agent to be incorporated into an aqueous gelatin solution during the production of various encapsulation compositions without adversely affecting the stability of the resulting encapsulation compositions. The rapid-release agent is further selected to dissociate upon contacting an aqueous solution having a pH ranging from about 0 to about 3, including, but not limited to, gastric juices.

The rapid-release agents release a gas including, but not limited to, carbon dioxide as a by-product of the dissociation of the rapid-release agent upon contact with gastric juices. Without being bound to any particular theory, the dissociation of the rapid-release agent releases gas bubbles within the coating composition. The hydrostatic pressure of the gas bubbles released by the dissociation of the rapid-release agent in the encapsulation exerts physical stresses on the surrounding polymer material of the encapsulation, causing the tearing and ultimate rupture of the encapsulation. The disruptive forces of the gas bubbles released by the dissociation of the rapid-release agent induces a significantly more rapid release of an active compound encapsulated by the rapid-release encapsulation composition compared to a composition that lacks the rapid-release agent.

In order to inhibit the formation of cross-bridges within the encapsulation during storage at conditions including but not limited to elevated temperature, elevated humidity, and combinations thereof, the encapsulation may further include hydrolysates of gelatin. Because the formation of cross-bridges in the encapsulation may hamper the dissolution of the encapsulation, the addition of gelatin hydrolysates may maintain the initial dissolution characteristics of the encapsulation, even after extended storage periods. In an exemplary embodiment, the hydrolysates of gelatin included in the rapid-release encapsulation composition have molecular weights ranging from about 100 to about 2000 Daltons.

Uses of the encapsulation composition include, but are not limited to, tablet coatings, soft gel-caps, and hard capsules. Other uses of the encapsulation composition embodiments may include, but are not limited to, chewable compositions and chewing gum that includes an encapsulated active therapeutic compound.

A more detailed description of various aspects of the invention is presented below.

### II. Rapid-Release Agents

Suitable rapid-release agents may include any compound capable of dissociating in an aqueous solution at a pH ranging from 0 to about 3. The suitable rapid-release agents may additionally release a gas including, but not limited to, carbon dioxide while dissociating in the aqueous solution at a pH of 0 to about 3.

Suitable rapid-release agents may include, but are not limited to bicarbonate and carbonate salts. The rapid-release agent may be a carbonate salt of an alkali metals or an alkaline earth metal including, but not limited to, lithium carbonate, sodium carbonate, potassium carbonate, rubidium carbonate, cesium carbonate, beryllium carbonate, magnesium carbonate, calcium carbonate, strontium carbonate, barium carbonate, and combinations thereof. The rapid-release agent may also be a carbonate salt of a transition element including, but not limited to, manganese carbonate, iron (II) carbonate, cobalt carbonate, nickel (II) carbonate, copper (II) carbonate, zinc carbonate, cadmium carbonate, and combinations thereof. The rapid-release agent may additionally be a carbonate salt of another metal including, but not limited to, thallium (I) carbonate, lead (II) carbonate, bismuth subcarbonate, and combinations thereof. The rapid-release agent may further be a carbonate salt of a lanthanide including, but not limited to, lanthanum carbonate.

Suitable rapid-release agents may be selected to be any compounds that are essentially insoluble in an aqueous solution at a pH ranging from about 6 to about 8, in addition to being soluble in an aqueous solution at a pH ranging from 0 to about 3. Suitable rapid-release agents having these solubility characteristics may be carbonate salts including, but not limited to, bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof. In particular, the rapid-release agent may be calcium carbonate.

For various edible products that may include rapid-release encapsulation compositions including but not limited to foods, dietary supplements, and pharmaceutical products, the rapid-release agents may be of at least food grade quality. More preferably, the rapid-release agents may be of GRAS and USP quality.

The rapid-release agents may be utilized in the form of fine particles less than about 0.152 mm (about 100 mesh) in size. The fine particles may be less than about 0.089 mm (about 170 mesh), less than about 0.075 mm (about 200 mesh), less than about 0.066 mm (about 230 mesh), or less than about 0.053 mm (about 270 mesh) in size. In particular, the rapid-release agents may be utilized in the form of fine particles less than about 0.075 mm (about 200 mesh) in size.

The amount of rapid-release agent included in the rapid-release encapsulation compositions may be sufficiently high to induce the formation of gas bubbles such as carbon dioxide when the encapsulation is exposed to an acidic solution such as gastric juices. Rapid-release agent may be included in the rapid-release encapsulation compositions in an amount ranging from about 5% to about 50% of the total weight of the composition. Alternatively, the amount of rapid-release agent included in the rapid-release encapsulation compositions may range from about 5% to about 13%, from about 9% to about 17%, from about 10% to about 18%, from about 14% to about 22%, from about 18% to about 26%, from about 22% to about 30%, from about 26% to about 34%, from about 30% to about 36%, from about 34% to about 40%, from about 38% to about 44%, from about 42% to about 48%, and from about 46% to about 50% of the total weight of the composition. In one embodiment, the amount of rapid release agent included in the rapid-release encapsulation compositions comprises about 5% to about 40% based on the weight of the gelatin. In another embodiment, the amount of rapid release agent included in the rapid-release encapsulation compositions comprises about 10% to about 30% based on the weight of the gelatin. In a further embodiment, the amount of rapid release agent included in the rapid-release encapsulation compositions comprises about 15% to about 20% based on the weight of the gelatin.

Preferably, the amount of rapid-release agent included in the rapid-release encapsulation composition may be just sufficient to induce the formation of bubbles when the composition is contacted with an acidic solution such as gastric juices. Higher proportions of rapid-release agents such as calcium carbonate may result in an encapsulation composition with undesirably brittle material properties. Further, an encapsulation composition with a relatively high proportion of rapid-release agent such as calcium carbonate may be vulnerable to the formation of undesired films during the production of the encapsulant. In particular, calcium carbonate may be included in the rapid-release encapsulation composition in the amount of about 15% by weight.

### III. Gelatin and Other Polymers

In addition to the rapid-release agent, the rapid-release encapsulation compositions will include a gelatin. The gelatin may be derived from collagen or collagen rich tissue including, but not limited to, the skin and bones of pigs or cattle. Non-limiting examples of gelatin include Type A gelatin, Type B gelatin and combinations thereof. Type A gelatin is characterized by an isoionic point ranging from about 7 to about 10.0, and is typically derived from collagen using an acid pretreatment method known in the art. Type B gelatin is characterized by an isoionic point ranging from about 4.8 to about 5.8.

The gelatin may typically include from about 80% to about 90% by weight protein, from about 0.1% to about 2% by weight mineral salts and from about 10% to 15% by weight water. “Protein”, as defined herein, refers to organic compounds made up of a plurality of amino acids joined together by peptide bonds between the carboxyl and amino groups of each adjacent amino acid. The gelatin may have an average molecular weight ranging from about 50,000 Da to about 300,000 Da. In another embodiment, the gelatin has an average molecular weight ranging from about 70,000 Da to about 150,000 Da. In a further embodiment, the gelatin has an average molecular weight ranging from about 80,000 Da to about 120,000 Da. Additionally, the gelatin typically comprises a Bloom value from about 50 to about 300. In one embodiment, the gelatin comprises a Bloom value ranging from about 125 to about 200. In yet another embodiment, the Bloom value may range from about 150 to about 175. The gelatin typically has a pH from about 3.8 to about 7.5. In another embodiment, the gelatin has a pH ranging from about 6.2 to about 7.3. In a further embodiment, the gelatin comprises a pH ranging from about 6.6 to about 7.0. The gelatin may also comprise an isoelectric point from about 4.7 to about 9.0, a viscosity from about 15 to about 75 mP and the ash content ranging from about 0.1% to about 2.0% by weight.

If the gelatin is substantially Type A gelatin, the bloom strength may range from about 50 to about 300, the pH may range from about 3.8 to about 5.5, the isoelectric point may range from about 7.0 to about 9.0, the viscosity may range from about 15 to about 75 mP and the ash content may range from about 0.1% to about 2.0% by weight.

If the gelatin is substantially Type B gelatin, the bloom strength may range from about 50 to about 300, the pH may range from about 5.0 to about 7.5, the isoelectric point may range from about 4.7 to about 5.8, the viscosity may range from about 20 to about 75 mP and the ash content may range from about 0.5% to about 2.0% by weight.

The gelatin may optionally be deionized prior to use by known methods including, but not limited to, ion exchange using a mixed bed of ion-exchange resin. The gelatin may also include gelatin hydrolysates having molecular weights ranging from about 100 Da to about 2000 Da. The gelatin hydrolysates and methods of producing the hydrolysates are described in U.S. Pat. No. 7,485,323, which is hereby incorporated by reference in its entirety.

The physical properties of the gelatin can and will vary depending upon its intended use. If the gelatin is to be used in the manufacture of hard capsule pharmaceutical products, the gelatin may have a bloom strength ranging from about 200 to about 300, a viscosity ranging from about 40 to about 60 mP and a pH ranging from about 4.5 to about 6.5. If the gelatin is to be used in the manufacture of soft shell capsule pharmaceutical products, the gelatin may have a bloom strength ranging from about 125 to about 200, a viscosity ranging from about 25 to about 45 mP and a pH ranging from about 4.5 to about 6.5.

The particular gelatin of the rapid-release encapsulation composition may be selected to possess an isoelectric point of below about 7.5. Because the addition of rapid-release agents may result in a composition pH that is significantly higher than previous gelatin encapsulation compositions, lower gelatin isoelectric points reduce the likelihood of occurrence of adverse chemical processes including but not limited to gelatin deamidation during the production of the rapid-release encapsulation composition.

In particular, the rapid-release encapsulation composition may include most typical Type B gelatins. Non-limiting examples of specific gelatins suitable for the rapid-release encapsulation compositions include acid bone gelatin, limed bone gelatin, and bovine hide gelatin.

The viscosity of the gelatin suspension used to produce the rapid-release encapsulation composition may be at a level at which the rapid release agents may not remain suspended during production. In these cases, an additional thixotropic compound including but not limited to a hydrocolloid such as hydroxyethyl cellulose or carboxymethyl cellulose may be added to the rapid-release encapsulation composition in order to maintain a sufficiently high viscosity of the gelatin suspension during production of the rapid-release encapsulation composition.

In addition, the term gelatin or gelatin component may be interpreted to encompass combinations of gelatin and other formulation additives such as plasticizers, aqueous solvents or mediums, and other components known in the art. The term plasticizer (also known as dispersants) is generally used to describe additives that impart increased flexibility and pliability to the gelatin formulation. Specifically, the plasticizer may be hydrophilic such as triethyl citrate and polyethylene glycol and/or hydrophobic such as diethyl phthalate, dibutyl phthalate, dibutyl sebacate and acetyl tributyl citrate. One skilled in the art will understand that other materials may be substituted for the polymer/plasticizer if they are capable of fulfilling the same function, i.e. imparting an increased level of flexibility and pliability to the gelatin formulation. It is possible that various hydrophobic materials including oils and waxes may also be used in this regard and can be found through routine experimentation or in the literature known to the skilled artisan. In one embodiment, the plasticizer comprises dibutyl sebacate, diethyl phthalate, glycerine, polyethylene glycol, propylene glycol, sorbitol, triacetin, and triethyl citrate, and mixtures thereof. In another embodiment, the plasticizer is selected from the group consisting of glycerine, sorbitol, erythritol, and combinations thereof. In a further embodiment, the plasticizer comprises a 1:1 mixture of glycerine and sorbitol.

The term aqueous solvent or aqueous medium may be interpreted to encompass any solvent capable of forming a gelatin composition. The aqueous medium includes, but is not limited to water.

In one embodiment, the gelatin component of the rapid-release encapsulation composition comprises a combination of gelatin, a plasticizer, and an aqueous medium comprising water. The gelatin component may include from about 35% to about 60% gelatin by weight of the gelatin component, from about 15% to about 30% plasticizer by weight of the gelatin component, and from about 25% to about 40% water by weight of the gelatin component. In another embodiment, the gelatin component comprises from about 42% to about 48% gelatin by weight of the gelatin component, from about 20% to about 25% plasticizer by weight of the gelatin component, and from about 30% to about 35% water by weight of the gelatin component.

In an additional embodiment, the gelatin component of the rapid-release encapsulation composition comprises a combination of gelatin and an aqueous medium comprising water. The gelatin component may include from about 5% to about 30% gelatin by weight of the gelatin component and from about 70% to about 95% water by weight of the gelatin component. In another embodiment, the gelatin component comprises from about 10% to about 20% gelatin by weight of the gelatin component and from about 80% to about 90% water by weight of the gelatin component.

In another embodiment, the gelatin component of the rapid-release encapsulation composition comprises a combination of gelatin and a gelatin hydrolysate, as previously described. The weight ratio of the gelatin to the gelatin hydrolysate generally ranges from about 3:1 to about 99:1. In another embodiment, the weight ratio of the gelatin to the gelatin hydrolysate ranges from about 4:1 to about 49:1. In yet another embodiment, the weight ratio of the gelatin to the gelatin hydrolysate ranges from about 5:1 to about 19:1.

The rapid-release encapsulation compositions may further include a polymer. The polymer may include any suitable encapsulating polymer known in the art including, but not limited to, synthetic polyvinyl polymers, synthetic polyethylene polymers, synthetic acrylic polymers, biopolymers, modified biopolymers, and combinations thereof. Suitable synthetic polyvinyl polymers include but are not limited to polyvinylchloride, polyvinylacetate and copolymers thereof, polyvinylalcohol, and polyvinylpyrrolidone. Synthetic polyethylene polymers may include but are not limited to polyethylene and polystyrene. Synthetic acrylic polymers may include but are not limited to methylmethacrylate or copolymers of acrylic monomers. Non-limiting examples of biopolymers and modified biopolymers include ethylcellulose, cellulose acetate phthalate, cellulose acetate, hydroxypropyl cellulose, hydroxypropylmethyl cellulose, methylcellulose, microcrystalline cellulose, carboxymethyl cellulose, Na-carboxymethyl cellulose, shellac, and gelatin.

For purpose of fabricating various encapsulation embodiments using an aqueous solution of encapsulation ingredients, a water-soluble polymer may be suitable for various embodiments, since many encapsulations including, but not limited to, tablet coatings, soft gelatin capsules, and hard capsules are formed from a liquid polymer solution. The water-soluble polymers may be soluble in aqueous solution at a pH ranging from about 6 to about 8. Non-limiting examples of water-soluble polymers include carboxymethylcellulose, cross-linked polyvinylpyrrolidone, hydroxylpropylcellulose, hydroxypropyl methylcellulose, microcrystalline cellulose, shellac, and gelatin. In particular, the rapid-release encapsulation composition may include gelatin and carboxymethylcellulose.

### IV. Method of Producing Rapid-Release Encapsulating Compositions

The rapid-release encapsulating composition that includes a rapid-release agent and a gelatin may be produced by dissolving the gelatin in water in a swelling process known in the art to form an aqueous solution ranging from about 5% to about 60% gelatin by weight. Alternatively, the aqueous gelatin solution may range from about 5% to about 15%, from about 10% to about 20%, from about 15% to about 25%, from about 20% to about 30%, from about 25% to about 35%, from about 30% to about 40%, from about 35% to about 45%, from about 40% to about 50%, from about 45% to about 55%, or about 50% to about 60% gelatin by weight. In particular, the aqueous solution may be about 15% gelatin by weight.

The gelatin may have varying particle sizes prior to the addition of the gelatin to the water to form the aqueous solution. In one embodiment, the gelatin particle sizes may vary from about 0.1 mm to about 10 mm. In other embodiments, the gelatin particle size may range from about 0.1 to about 0.3 mm, from about 0.2 to about 0.8 mm, from about 0.5 to about 1.5 mm, from about 1 to about 3 mm, from about 2 to about 6 mm, or from about 5 to about 10 mm. Without being bound to any particular theory, the particle size of the gelatin may impact the amount of time needed for the gelatin to degrade in aqueous solution. Gelatins having a particle size ranging from about 0.1 to about 0.3 mm may swell in solution within a few minutes, gelatins having a particle size ranging from about 0.3 to about 0.8 mm may swell in solution within a time from about 8 to about 12 minutes, and gelatins having a particle size greater than about 0.8 mm may swell within about an hour.

Gelatin solutions having a concentration ranging from about 10% to about 20% by weight of gelatin may be prepared using any gelatin particle size. In another embodiment having a more concentrated solution ranging from about 30% to about 34% gelatin by weight, gelatin particles larger than about 0.8 mm in size may be used to inhibit aggregation and air bubble formation during processing.

The pH of the gelatin solutions may be adjusted to a pH ranging from about 6 to about 8 by the addition of an acid or base. In one embodiment, the gelatin solution is adjusted to a pH level ranging from about 6.6 to about 7.0 prior to addition of the rapid-release agent. In a further embodiment, the pH of the gelatin solution is adjusted to about 6.8, prior to addition of the rapid-release agent. In those embodiments in which the encapsulant is to be used for pharmaceutical encapsulant applications, suitable acids may include food grade acids. Non-limiting examples of suitable food-grade acids include sulfuric acid, tartaric acid, citric acid, acetic acid, and carbon dioxide gas from carbon dioxide sources including, but not limited to, dry ice, phosphoric acid, or combinations thereof. Non-limiting examples of suitable food-grade bases include sodium hydroxide, potassium hydroxide, calcium hydroxide, sodium bicarbonate, potassium bicarbonate, calcium oxide or combinations thereof. Without being bound to any particular theory, the adjustment of the pH may alter the cross-linking or ionic interactions of gelatin molecules, thereby altering the material properties of the resulting gelatin encapsulant material including, but not limited to, hardness, solubility in aqueous solution having a low pH ranging from 0 to about 3, and combinations thereof.

The rapid-release agent may be added to the aqueous solution of gelatin, forming an encapsulation composition having a mass ratio of the rapid-release agent to the gelatin ranging from about 1:1 to about 1:20. The mass ratio is defined as the ratio of the mass of rapid-release agent compared to the mass of the gelatin solution, wherein the gelatin solution comprises the combined mass of the gelatin, the aqueous solution in which the gelatin is dissolved, as well as the mass of any gelatin hydrolysate incorporated into the solution. Alternatively, the mass ratio of the rapid-release agent to the gelatin may range from about 1:1 to about 1:8, from about 1:4 to about 1:10, from about 1:6 to about 1:12, from about 1:8 to about 1:14, from about 1:10 to about 1:16, from about 1:12 to about 1:18, or from about 1:14 to about 1:20 in the encapsulation composition. In one embodiment, the mass ratio of the rapid-release agent to the gelatin may range from about 1:2 to about 1:15 in the encapsulation composition. In a further embodiment, the mass ratio of the rapid-release agent to the gelatin may range from about 1:4 to about 1:9 in the encapsulation composition.

The rapid-release agent is essentially insoluble in the aqueous solution of gelatin. Without being bound to any particular theory, the addition of the rapid-release agent may not significantly alter any of the chemical properties of the encapsulation composition including, but not limited to, pH that may induce changes in the cross-linking or ionic interactions of the gelatin molecules in the encapsulation composition. In some embodiments, the rapid-release agent may alter the pH of the gelatin solution slightly but the magnitude of the change may not be sufficient to cause undue instability of the gelatin to hydrolysis.

### V. Exemplary Encapsulant Compositions

The rapid-release encapsulation composition includes a rapid-release agent described in Section II above and a gelatin described in Section III above. The rapid-release encapsulation composition may be produced using the method described in Section IV above.

One exemplary rapid-release encapsulation composition includes gelatin in an aqueous solution containing about 15% gelatin by weight, and from about 2% to about 10% sodium bicarbonate by weight. Another exemplary rapid-release encapsulation composition includes gelatin in aqueous solution containing about 15% gelatin by weight, and from about 5% to about 15% calcium carbonate by weight. The particular composition of the gelatin in any of the exemplary embodiments may vary depending upon the intended use of the encapsulation composition, as described in Section III above.

A further exemplary rapid-release encapsulation composition includes gelatin in an aqueous solution containing from about 10% to about 15% gelatin by weight, from about 5% to about 15% calcium carbonate by weight, and from about 1% to about 5% gelatin hydrolysate by weight. The particular composition of the gelatin in any of the exemplary embodiments may vary depending upon the intended use of the encapsulation composition, as described in Section III above.

An additional exemplary rapid-release encapsulation composition includes gelatin in an aqueous solution containing from about 36% to about 42% gelatin by weight, from about 17% to about 22% plasticizer by weight, from about 26% to about 31% water by weight, and from about 10% to about 15% calcium carbonate by weight. The particular composition of the gelatin in any of the exemplary embodiments may vary depending upon the intended use of the encapsulation composition, as described in Section III above.

Another exemplary rapid-release encapsulation composition includes gelatin in an aqueous solution containing from about 32% to about 40% gelatin by weight, from about 17% to about 22% plasticizer by weight, from about 26% to about 31% water by weight, from about 10% to about 15% calcium carbonate by weight, and from about 2% to about 6% gelatin hydrolysate by weight. The particular composition of the gelatin in any of the exemplary embodiments may vary depending upon the intended use of the encapsulation composition, as described in Section III above.

### VI. Therapeutic Compositions

Any of the rapid-release encapsulation compositions described above may be used in the production of a variety of therapeutic compositions that include the rapid-release encapsulation composition. Non-limiting examples of therapeutic composition embodiments include rapid-release coated tablets, soft-gel capsules, hard capsule shells, chewable therapeutic compositions including, but not limited to, chewable antacid compositions, and a chewing gum containing encapsulated active compounds including, but not limited to, a chewable antacid composition.

The various therapeutic compositions include an active pharmaceutical ingredient (API) in addition to the rapid-release encapsulant composition. The active pharmaceutical ingredient (API) may be selected from groups of APIs including, but not limited to, abortifacients, ACE inhibitors, adrenocorticotropic hormones, α-adrenergic agonists, α-adrenergic blockers, α-glucosidase inhibitors, anabolic steroids, narcotic analgesics, non-narcotic analgesics, anorexics, antacids, antihelmintics, antiallergics, antialopecials, antiamoebics, antianginals, antiarrhythmics, antiarthritics, antiasthmatics, antibiotics, anticholinergics, anticonvulsants, antidepressants, antidiabetics, antidiarrheals, antidotes, antidyskinetics, antiemetics, antiestrogens, antifungals, antiglaucoma agents, antigout agents, antihistaminics, antihypertensives, nonsteroidal antiinflamatories, antimalarials, antimigraines, antimuscarinics, antinauseants, antineoplastics, antiparkinsonians, antipheochromocytoma agents, antipneumocystis, antiprostatic hyperplasia agent, antiprotozoals, antipruritics, antipsoriatics, antipsychotics, antipyretics, antirickettsials, antispasmodics, antithrombocythemics, antithrombotics, antithyroid agents, antituberculosis agents, antitussives, antiulceratives, antivirals, anxiolytics, aromatase inhibitors, autonomic drugs, barbiturates, benzodiazepine antagonists, β-adrenergic antagonists, β-adrenergic blockers, bradycardic agents, bronchodilators, calcium channel blockers, carbonic anhydrase inhibitors, cardiac drugs, cardiotonics, choleretics, cholinergics, cholinesterase inhibitors, cholinesterase reactivators, CNS stimulants, cytoprotectants, decongestants, diuretics, dopamine receptor agonists, dopamine receptor antagonists, ectoparasiticides, emetics, expectorants, fibrinogen receptor antagonists, gastric secretion inhibitors, gastrointestinal drugs, gastroprokinetics, genitourinary smooth muscle relaxants, heavy metal antagonists, hemostatics, histamine H2 receptor antagonists, hypnotics, immunomodulators, immunosuppressants, iron preparations, keratolytics, MAO inhibitors, mucolytics, muscle relaxants, mydriatics, narcotic antagonists, nootropics, opiate agonists, oxytocics, potassium channel activators, respiratory stimulants, sedatives, serenics, serotonin receptor agonists, serotonin receptor antagonists, serotonin uptake inhibitors, stimulants, sympatholytic agents, sympathomimetics, thrombolytics, tocolytics, tranquilizers, vasodilators, vasoprotectants, and vitamins.

The rapid-release therapeutic compositions may include but are not limited to APIs that are water-soluble. The APIs may be water-soluble in aqueous solutions having a pH ranging from 0 to about 9. Alternatively, the water-soluble APIs may be water-soluble in aqueous solutions having a pH ranging from 0 to about 3, including, but not limited to, gastric juices. Non-limiting examples of water soluble APIs include abacavir sulfate, acebutolol, acetaminophen, acyclovir, albendazole, alendronate sodium, allopurinol, amoxicillin, amantadine HCl, aminobenzoate potassium, aminocaproic acid, aminoarone HCl, amitriptyline hydrochloride, amphetamine, aspirin, atenolol, atorvastatin calcium, atropine sulfate, azithromycin, balsalazide, benzepril hydrochloride, bepridil HCl, betaine HCl, bisoprolol fumarate, buformin, bupropion HCl, calacyclovir, capecitabine, captopril, carisoprodol, cefadroxil, cefdnir, cefixime, cefpodoxime proxetil, cefprozil, cefuroxime axetil, celecoxib, cetrizine hydrochloride, chondroitin, chlorathiazide, chlorpheniramine maleate, chlorpromazine HCl, chlorzoxazone, choline magnesium trisalicylate, cimetidine, ciprofloxacin, clavulanate potassium, clindamycin, clomipramine hydrochloride, clonidine hydrochloride, clopidogrel bisulfate, cloxacillin sodium, codeine phosphate, colchicines, colsevelam HCl, creatine, cyclophosphamide, cyproheptadine, delavirdine mesylate, demeclocycline HCl, diclofenac, didanosine, diethylcarbamazine citrate, diltiazem HCl, DL-methionine, doxepine HCl, doxycycline, efavirenz, eprosartan mesylate, entacapone, ethembutol hydrochloride, eprosartan, erythromycin, ethosuximide, etidronate disodium, etodolac, ferrous sulfate, flecamide acetate, felbamate, fexofenadine HCl, firocoxib, fluconazole, fluoxetine hydrochloride, fluriprofen, fluvastatin, fosonopril sodium, fumarate, gabapentine, gatifloxacin, ganciclovir, guaifenesine, hydralazine hydrochloride, hydrocodone bitartrate, hydroxychloroquine sulfate, hydroxyurea, hydroxyzine hydrochloride, ibuprofen, indinavir sulfate, irbesartan, isoflavone, isoniazid, isosorbide mononitrate, ketoprofen, lactobionate, lamivudine, levamisole hydrochloride, levofloxacin, lisinopril, lithium carbonate, losartan potassium, mebendazole, mefenamic acid, meperidine HCl, mesalamine, metaprolol tartrate, metaxalone, metformin HCl, methenamine mandelate, methdopa, methocarbamol, methylphenidate, methylphenidate hydrochloride, metyrosine, minocycline hydrochloride, modafinil, montelukast sodium, morphine sulfate, moxifloxacin HCl, mycophenolate mefetil, nabumetone, naproxen sodium, nefazodone HCl, nelfinavir meslyate, neostigmine bromide, niacin, nicotinamide, nitrofurantoin, nifurtimox, nizatidine, norfloxacin, nortriptyline hydrochloride, ofloxacin, olanzepine, orlistat, oxybytynin chloride, pancreatin, pantothenic acid, penicillamine, penicillin V potassium, pentosan polysulfate sodium, phenformin, phenylbutazone, phenyloin sodium, phytoestrogen, potassium chloride, pramipexole, pravastatin sodium, praziquantel, primaquine phosphate, proanthocyanidin, procainamide, promethazine, promethazine hydrochloride, propafenone HCl, propanolol HCl, propoxyphene hydrochloride, propoxyphene napsylate, prozosin, pseudophedrine hydrochloride, pseudoephedrine sulfate, psyllium, pycnogenol, pyrazinamide, pyridostigmine bromide, pyridoxine hydrochloride, pyruvate, quetiapine carafate, quinidine sulfate, quinapril hydrochloride, ramipril, ranitidine hydrochloride, reboxetine, rifabutin, rifampin, risedronate sodium, rofecoxib, rosiglitazone maleate, salbutamol sulfate, saquinavir mesylate, sertraline HCl, sevelamer HCl, sildenafil, simethicone, sodium valproate, sotalol HCl, stavudine, succimer, sumanirole, sumatriptan succinate, suntheanine, terazosin hydrochloride, terbinafine HCl, tetracycline HCl, theophylline, thiobendazole, ticlopidine HCl, timolol meleate, tocamide HCl, tolcapne, tolmetin sodium, tramadol HCl, trovafloxacin mesylate, valacyclovir HCl, valganciclovir HCl, valsartan, vancomycin, venlafaxine hydrochloride, verapamil HCl, warfarin sodium, xylamine, zidovudine, and combinations thereof. Depending on the particular embodiment, the API may be in a solid, powder, particulate, or liquid form.

### A. Coated Tablets

One therapeutic composition may be a rapid-release tablet coating composition that includes the rapid-release encapsulation composition in which the encapsulation composition is applied as a thin coat over the outer surface of an API in solid tablet form. The encapsulation composition may be applied to the API using any technique known in the art, including, but not limited to, pan coating, drum coating, film coating, spray coating, and dip coating.

### B. Soft-Gel Capsules

Another therapeutic composition may be a rapid-release soft-gel capsule composition that includes the rapid-release encapsulation composition in which the encapsulation composition forms a continuous membrane that encloses the API, which may be in a liquid or powder form. The encapsulation composition may be formed into a gel-cap using any technique known in the art, including, but not limited to, forming and filling individual gel-caps in a mold, formation of the gel-caps using a rotary die and filling using blow molding, or Accogel-type encapsulation techniques. In these embodiments, the encapsulation composition may further include a plasticizer including but not limited to glycerin, mixtures of sorbitol derivatives or mixtures thereof.

### C. Hard Capsules

An additional therapeutic composition may be a hard capsule composition that includes the rapid-release encapsulation composition in which the encapsulation composition forms a rigid shell that encloses the API, which may be in a liquid, granular, or powder form. The hard capsule composition may be in a form including, but not limited to, a continuous shell formed around the API, or two telescopically-joined half-shells in which each half-shell is formed separately, and the API is inserted prior to joining the half-shells. The encapsulation composition may be formed into a hard capsule using any technique known in the art, including, but not limited to, dip-coating metal rod ends, or injection molding.

### D. Chewable Active Compounds

Yet another therapeutic composition may be a chewable therapeutic composition that includes the rapid-release encapsulation composition in which the encapsulation composition may coat each of a plurality of small API particles. When contacted with acidic aqueous solutions including but not limited to gastric juices, the encapsulation rapidly degrades, causing the quick release of the API. One non-limiting exemplary therapeutic composition is a chewable antacid formulation in which the API is an antacid coated with the encapsulant composition. The coated API particles of the chewable active composition may be formed by any method known in the art including, but not limited to, spray coating, pan coating, drum coating, or emulsion coating. The chewable active composition may be formed into a hard tablet using techniques known in the art including direct compression, wet granulation, dry granulation, and fluidized bed granulation.

### E. Therapeutic Chewing Gum Composition

Still another therapeutic composition may be a therapeutic chewing gum composition that includes the rapid-release encapsulation composition in which the encapsulation composition may coat the small API particles. The encapsulation composition may form chewable gelatin capsules enclosing the API particles. When contacted with acidic aqueous solutions including but not limited to gastric juices, the encapsulation rapidly degrades, causing the quick release of the API. One non-limiting exemplary therapeutic composition is an antacid chewing gum composition in which the API is an antacid coated with the encapsulant composition. The coated API particles of the chewable active composition may be formed by any method known in the art including, but not limited to, spray coating, pan coating, drum coating, or emulsion coating. The chewable active composition may be formed using techniques known in the art including, but not limited to, mixing the encapsulated API into a panning syrup and applying the mixture as a gum coating using a panning technique.

### EXAMPLES

The following examples illustrate various aspects of the invention.

### Example 1

### Effect of Production Method on Dissolution Properties of Gelatin Encapsulant Compositions

To assess the effect of the addition of acid compounds used in the production of gelatin encapsulant compositions on the dissolution properties of the compositions, the following experiment was conducted.

600 grams of pigskin gelatin was dissolved in 3400 g of deionized water, and the mixture was filtered through a mixed bed of ion-exchange resin (pH>9). The gelatin mixture was then divided into five parts and adjusted to a pH of 5.5±0.1 using the following acids: sulfuric acid, tartaric acid, citric acid, acetic acid, and carbon dioxide. The carbon dioxide was supplied either by the sublimation of dry ice or using sodium bicarbonate. Each of the five mixtures was chilled overnight and then dried in dehydrators. In the case of treatment with carbon dioxide, a pH of 5.5 was not attained but the pH was significantly reduced from pH 9.

The dried samples were ground and prepared as a coating. A 30% gelatin solution of each sample was prepared by weighing 50 grams of the dried gelatin into a 250 ml beaker, adding 116.7 g of de-ionized water, and stirring to mix. After mixing, the beaker was covered with a watch glass and allowed to swell for about 1 hour at room temperature. The mixture was then melted at 60° C. for about 4 hours, stirring after about 1 hour stir to mix. A series of glass plates were preheated to a temperature of about 60° C. and loaded into an automated coating device. After removing any skin or bubbles from the surface of the melted gelatin mixture, the mixture was loaded into the automated coating device and coated on to the series of preheated glass plates. The films were stored overnight (approximately 17 hours) in a temperature and humidity controlled room at 45%±5% RH and 70°±5° F.

For each of the five compositions, a sample of the coating weighing approximately 0.075 g was placed into each of six reaction vessels filled with 900 g of KH₂PO₄ solution having a pH of 3.0 and heated to 37° C. The absorptivity of the reaction vessel contents at a wavelength of 218 nm was used to measure the dissolution of the coating samples over a period of about 15 minutes.

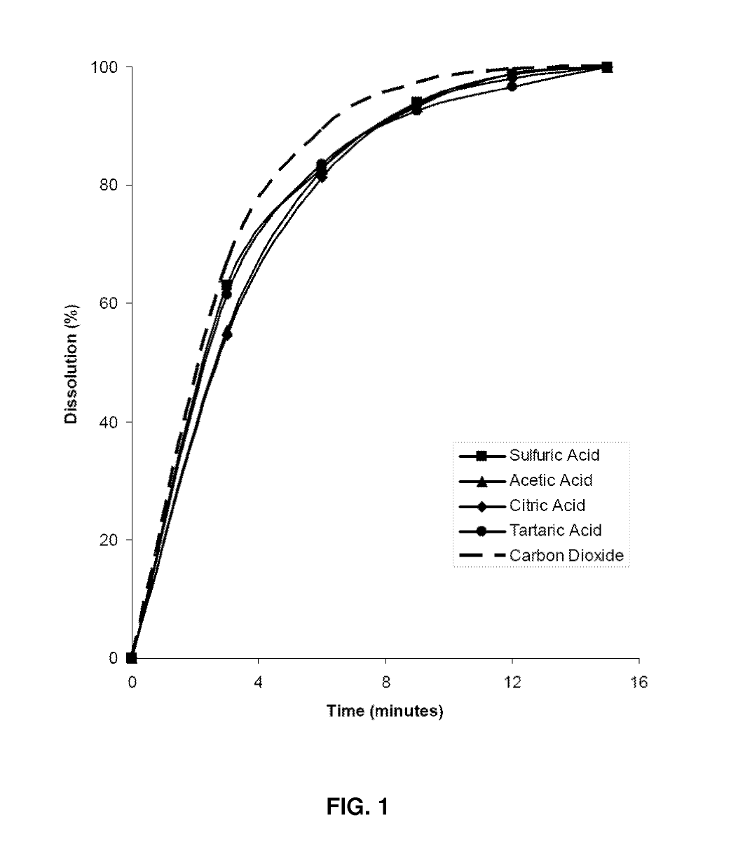

The results of the dissolution measurements are summarized in FIG. 1. The dissolution curve for the coating composition that incorporated carbon dioxide in the form of sodium bicarbonate or dry ice did not significantly affect the dissolution properties of this composition relative to any of the other coating compositions. However, in this experiment, sodium bicarbonate was not added to the coating composition in an amount sufficient to induce the formation of CO₂ gas bubbles during the dissolution of the composition.

The results of this experiment demonstrated that the dissolution rates of the coating compositions tested in this experiment were sensitive to the composition of the coating. In particular, the dissolution properties of the coating composition that incorporated carbon dioxide produced using dry ice or sodium bicarbonate in the amounts specified by this experiment were not significantly different than any of the other coating compositions tested.

### Example 2

### Effect of Sodium Bicarbonate Added During Production of Gelatin Encapsulation Compositions on Dissolution Properties

To assess the effect of adding sodium bicarbonate during the production of a gelatin encapsulation composition on the dissolution properties of the resulting gelatin coating, the following experiment was conducted.

600 grams of gelatin was dissolved in 3400 g of deionized water, and the mixture was filtered through a mixed bed of ion-exchange resin (pH>9). The gelatin mixture was then adjusted to a pH of 5.5±0.1 using sulfuric acid. The gelatin mixture was then divided into two halves, and 2% sodium bicarbonate by weight was added to one half of the gelatin mixture. Each of the two mixtures was chilled overnight and then dried in dehydrators. Coating samples were formed from the two mixtures using the methods described in Example 1.

A buffer solution having a pH of 1.0 was formed by adding 200 ml of 2M HCl and 29.8 g of KCl to 1800 ml of deionized water. Coating samples from the two gelatin mixtures were added to two sets of six reaction vessels containing 900 g of the pH=1 buffer solution and the dissolution of the coating samples was measured using the method previously described in Example 1.

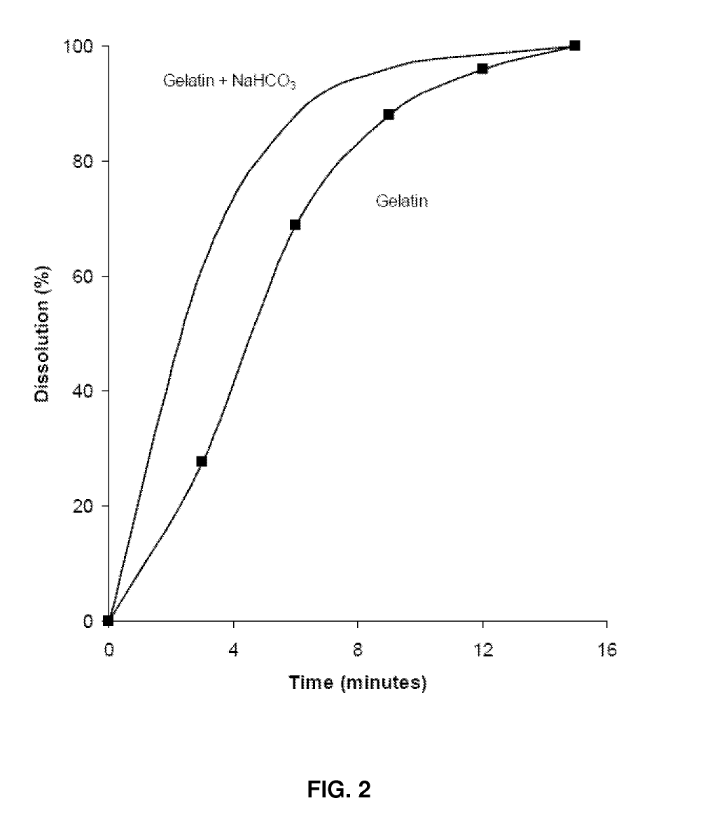

The measured dissolutions of the coating samples are summarized in FIG. 2. The addition of sodium bicarbonate to the gelatin mixture during the production of the gelatin encapsulant composition significantly increased the rate of dissolution of the resulting gelatin coating relative to the same gelatin coating produced without any added sodium bicarbonate. In particular, the addition of sodium bicarbonate significantly increased the rate of tearing apart of the film during the procedure.

The results of this experiment demonstrated that the addition of sodium bicarbonate during the production of a gelatin coating composition significantly increased the rate of dissolution of the resulting gelatin coating compared to the same gelatin coating composition that lacked sodium carbonate, largely due to the increased rate of tearing apart of the film due to carbon dioxide bubbles formed from the sodium carbonate during the procedure.

### Example 3

### Effect of Carbonate Compounds on pH of Gelatin Mixtures

To assess the sensitivity of the pH of a deionized gelatin suspension to the addition of various carbonate compounds, the following experiment was conducted. A deionized gelatin suspension was formed using the methods described in Example 1 and sulfuric acid was added to the gelatin suspension to adjust the pH of the suspension to about 4.7. The gelatin suspension was divided into three equal parts. To the first part of the gelatin suspension, 10% calcium carbonate by weight was added. To the second and third parts of the gelatin suspension, sodium bicarbonate in the amount of 5% and 7.5% by weight was added, respectively. The pH of each gelatin suspension was measured before and after the addition of the carbonate, and is summarized in Table 1 below:

TABLE 1
pH of Gelatin Suspensions Before
and After Addition of Carbonates

pH of Gelatin Suspension

| Carbonate Added to | | Before Addition | After Addition of |
|---|---|---|---|
| Suspension | | of Carbonate | Carbonate |
| 10% | CaCO₃ (wt %) | 4.7 | 7 |
| 5% | NaHCO₃ (wt %) | 4.7 | 7.59 |
| 7.5% | NaHCO₃ (wt %) | 4.7 | 7.75 |

The addition of the carbonates to the gelatin mixtures generally increased the pH of the gelatin suspension as expected due to the basic properties of carbonates in solution. However, the addition of calcium carbonate increased the pH of the gelatin suspension significantly less than the addition of sodium bicarbonate, despite adding a higher amount of calcium carbonate. This was most likely due to the lower solubility of the calcium carbonate at the original pH of the gelatin suspension (pH=4.7) compared to sodium bicarbonate. Because less calcium carbonate underwent dissociation in the gelatin mixture, less neutralization of the gelatin suspension occurred, resulting in the maintenance of a lower pH in the gelatin suspension after the addition of the calcium carbonate.

The results of this experiment demonstrated that the pH of the gelatin suspensions tested were sensitive to the amount and composition of the carbonate added to the suspension.

### Example 4

### Effect of Extended Storage at Elevated Temperature and Humidity on the Dissolution Properties of Gelatin Encapsulation Compositions

To assess the sensitivity of the dissolution properties of several gelatin encapsulation compositions to extended storage times at elevated temperature and humidity, the following experiment was conducted. A deionized bone gelatin suspension was formed and used as an encapsulation composition using methods similar to those described in Example 1. The gelatin suspension was divided into three equal parts and used to form three different encapsulation compositions. The first encapsulation composition included the bone gelatin without further modification (CONTROL). The second encapsulation composition included the bone gelatin as well as 15% CaCO₃ by weight (FD−fast-dissolving). The third encapsulation composition included the bone gelatin, 15% CaCO₃ by weight, and 10% by weight of hydrolyzed bone gelatin having a molecular weight of about 500 Daltons (FD+SH). The dissolution of each of the three encapsulation compositions in deionized water and in a pH=1 buffer solution were measured using the method described in Example 1. The dissolutions of the encapsulation compositions were measured immediately after the compositions were produced, as well as after storage at 50° C. and 80% relative humidity for periods of two, five and eleven weeks.

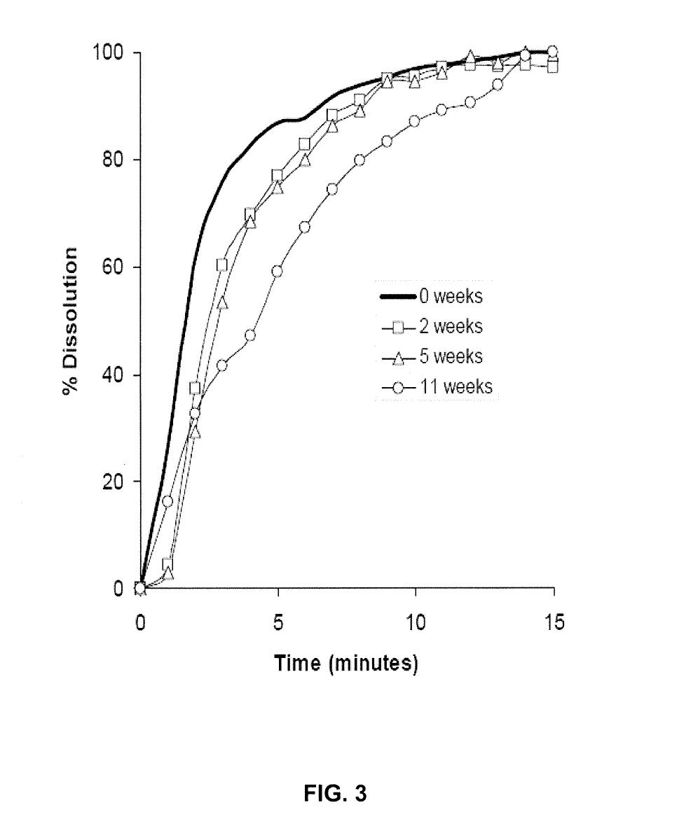

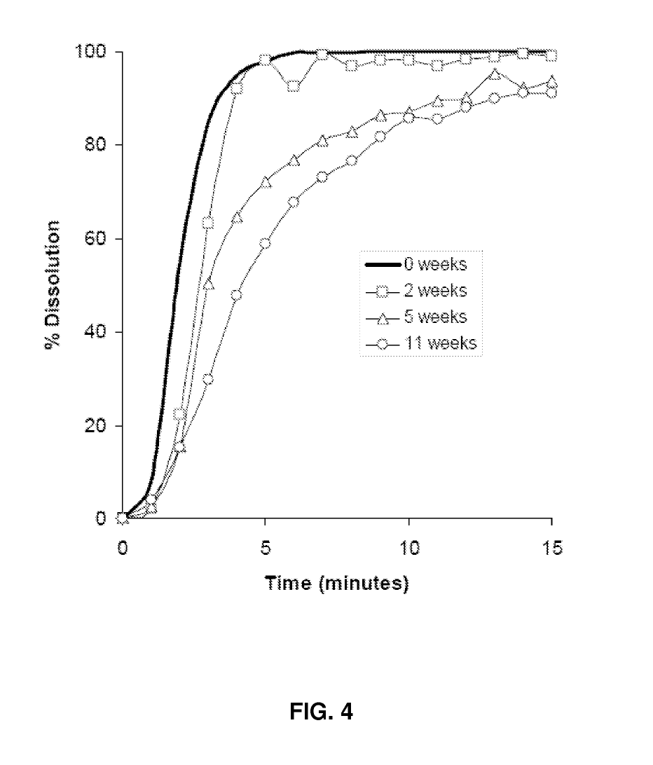

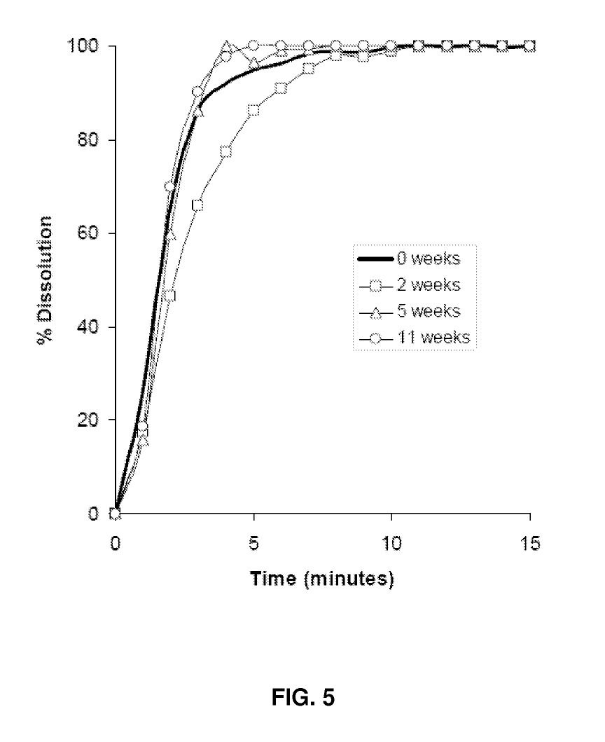

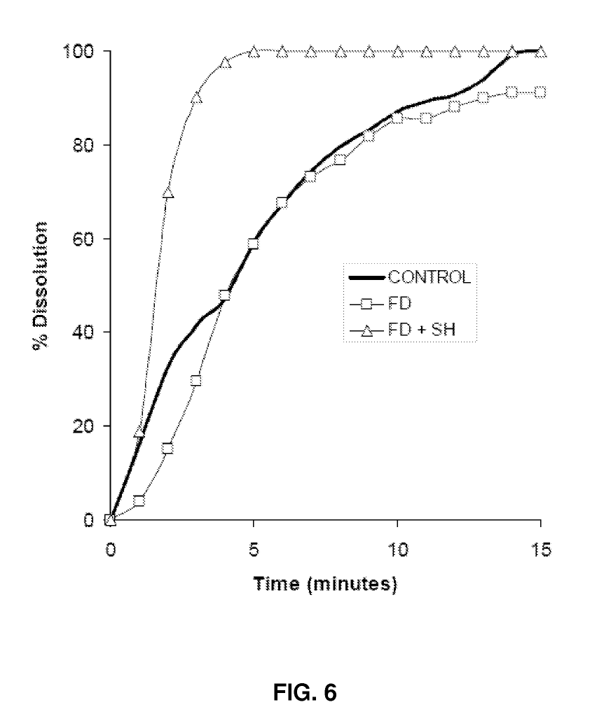

The dissolution results for the CONTROL, FD, and FD+SH encapsulation compositions in pH=1 buffer solution are summarized in FIGS. 3, 4, and 5 respectively. FIGS. 3 and 4 both show a marked reduction in the dissolution rate at longer periods of storage at 50° C. and 80% relative humidity. Although FIG. 5 exhibits a similar trend of reduction of dissolution rate for the FD+SH encapsulation composition after two weeks of storage, the dissolution rate returns to levels similar to initial dissolution rates after five and eleven weeks of storage. FIG. 6 is a comparison of the dissolution results of the three encapsulation compositions in pH=1 buffer solution after eleven weeks of storage. The dissolution rate of the FD+SH composition is maintained at a significantly higher level than either the CONTROL or FD compositions.

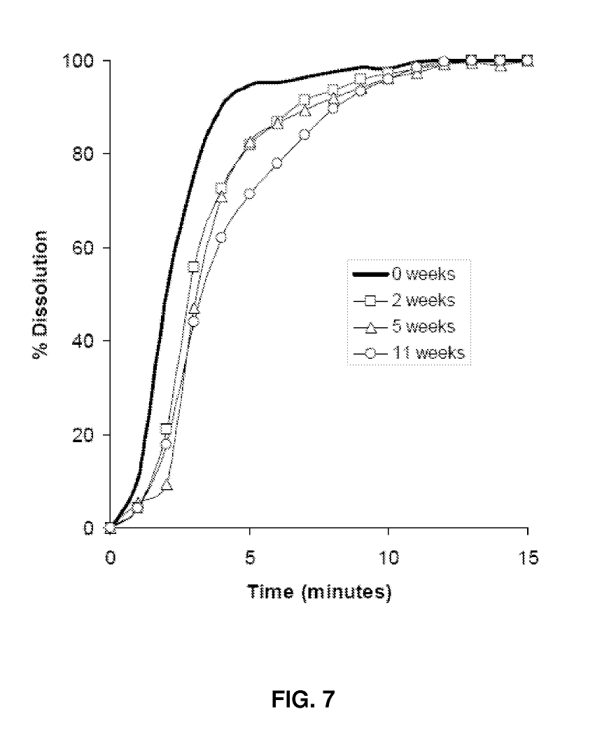

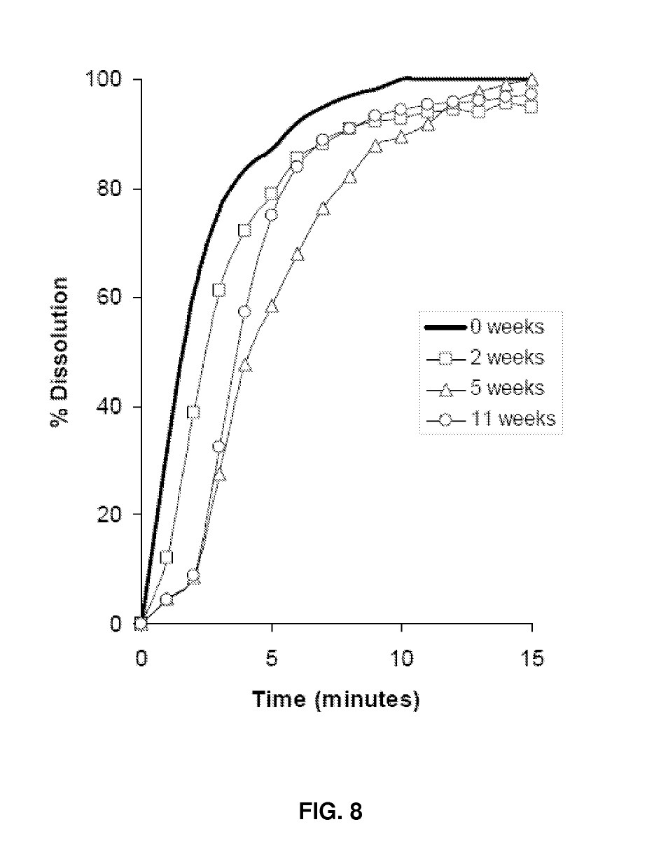

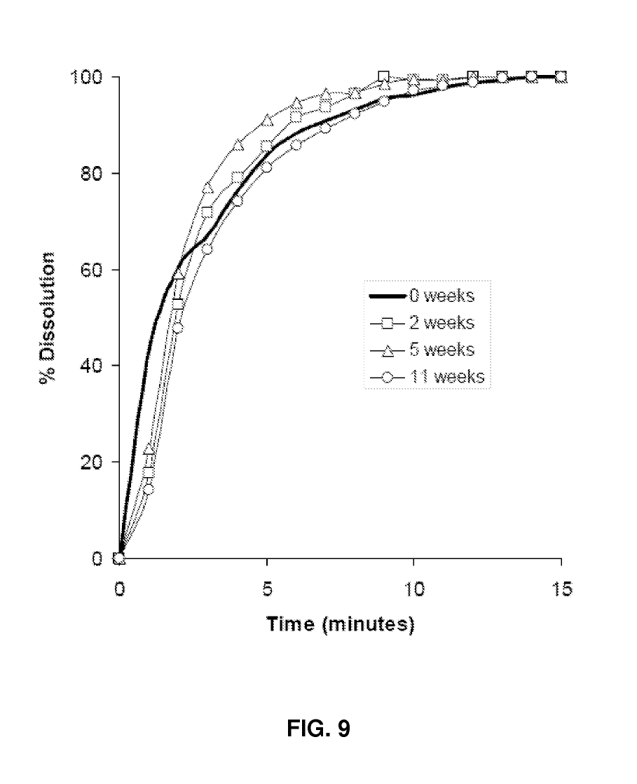

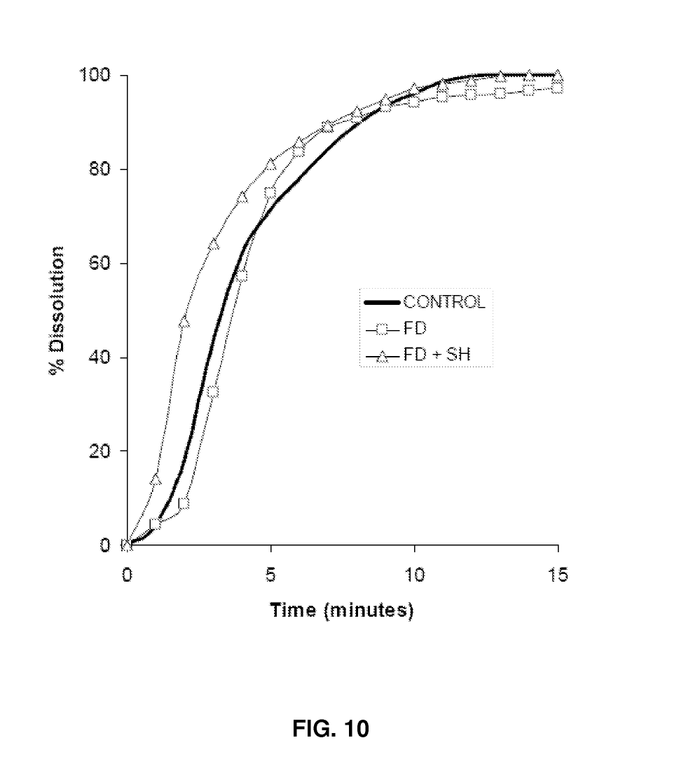

The dissolution results for the CONTROL, FD, and FD+SH encapsulation compositions in deionized water are summarized in FIGS. 7, 8, and 9 respectively. FIGS. 7 and 8 both show degradations of dissolution rate for the CONTROL and FD compositions after extended periods of storage in a manner similar to the degradations shown in FIGS. 3 and 4. As shown in FIG. 9, the dissolution rate of the FD+SH composition in deionized water was essentially unaffected by periods of extended storage at 50° C. and 80% relative humidity. FIG. 10 is a comparison of the dissolution results for the CONTROL, FD, and FD+SH compositions in deionized water after 11 weeks of storage, clearly showing that the FD+SH composition maintains a significantly higher dissolution rate even after eleven weeks of storage.

The reduction in dissolution rate in the CONTROL and FD encapsulation compositions after storage at elevated temperature and humidity conditions is likely due to the formation of cross-bridges within the gelatin in the encapsulation compositions. The addition of low molecular weight hydrolysates having molecular weights from about 100 to about 2000 Daltons to the encapsulation composition may interfere with the formation of cross-bridges, thereby maintaining the dissolution characteristics of the gelatin encapsulation compositions at initial levels, even after extended periods of storage.

The results of this experiment demonstrated that the dissolution properties of gelatin encapsulation compositions may degrade after storage at elevated temperatures and humidity. This degradation is sensitive to the particular composition, and the degradation may be virtually eliminated by the addition of hydrolysates having molecular weights from about 100 to about 2000 Daltons to the gelatin encapsulation compositions.

### Example 5

### Effect of Extended Storage at Elevated Temperature and Humidity on Dissolution Properties

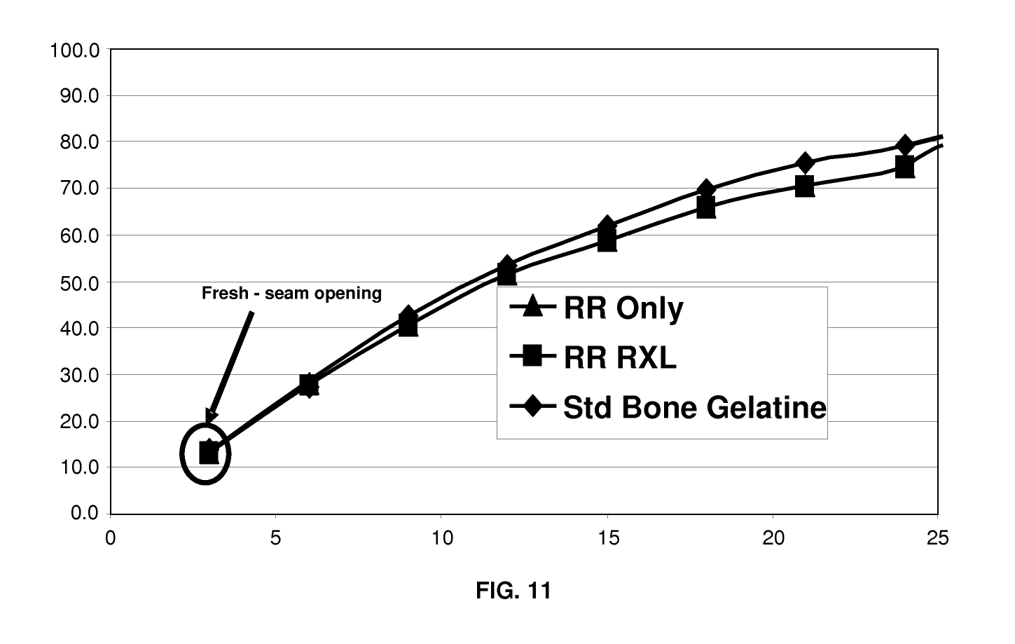

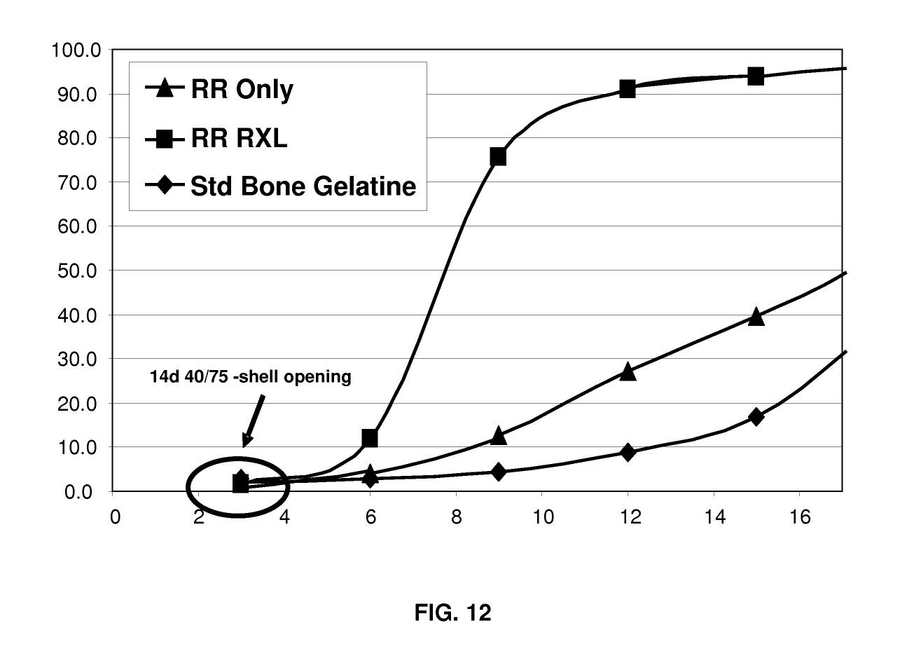

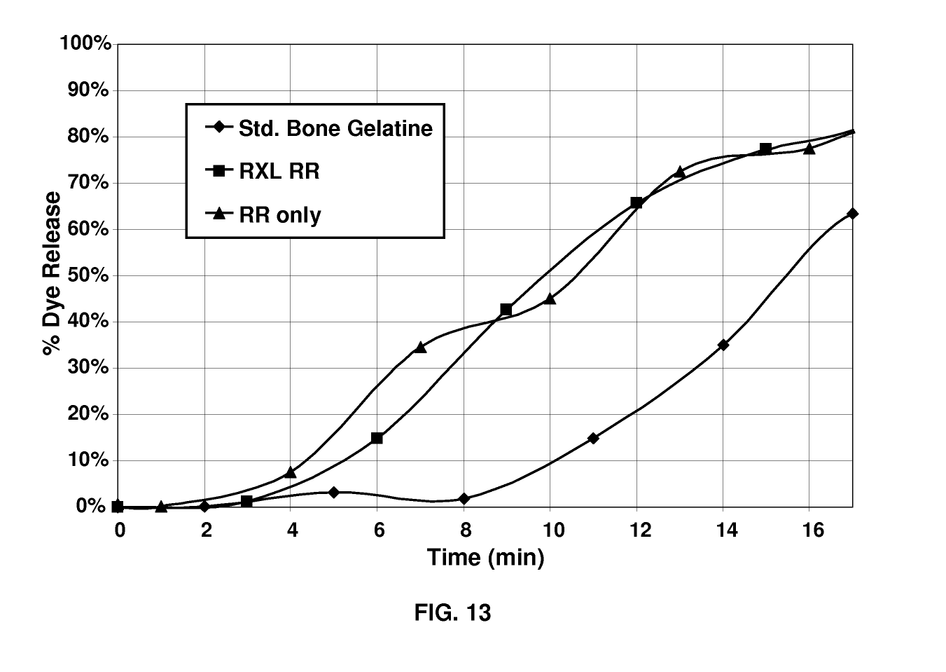

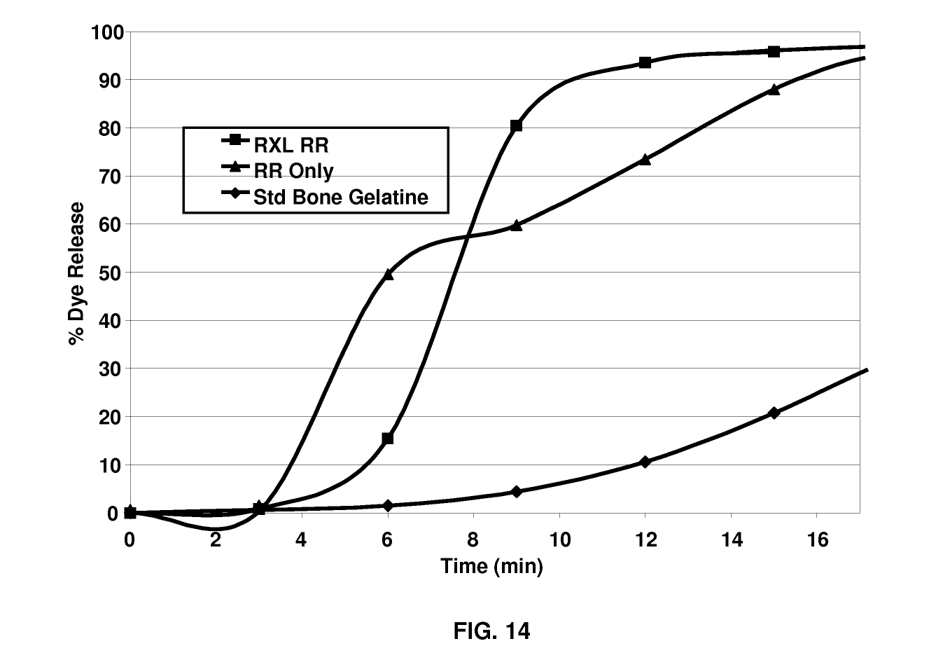

To further assess the dissolution properties of the various gelatin formulations discussed herein after exposure to temperature and humidity conditions, the following soft capsule experiment was conducted. Soft capsule encapsulation compositions were prepared having viscosities at 60° C. of about 10,000 mPas. The first encapsulation composition included the bone gelatin without any further modifications at 43.00 weight percent, sorbitol at 10.75%, glycerol at 10.75%, and water at 35.5% (“Std Bone Gelatine”). The second encapsulation composition included the bone gelatin at 40.8%, calcium carbonate (CaCO₃) at 7.2%, sorbitol at 10.2%, glycerol at 10.2%, and water at 31.6% (“RR only”). The third encapsulation composition included the bone gelatin at 38.35%, hydrolyzed bone gelatin having a molecular weight of about 500 Daltons at 2.45%, calcium carbonate (CaCO₃) at 7.2%, sorbitol at 10.2%, glycerol at 10.2%, and water at 31.6% (“RR RXL”). The three encapsulation compositions were utilized to make soft capsules on a Modified Chan Sung soft capsule machine Type M3 having dual cavity dye rolls to make 7.5 oval capsules and operating at 2.5 rpm with 30 minute tumble drying followed by one week room drying at about 25° C. and about 35% RH. The capsules were filled with a liquid formulation comprising polyethylene-glycol at 96.51%, glycerol at 2.99%, and brilliant blue dye at 0.50% (the fill). The three formulations were tested to determine the dissolution profile over time for three distinct dissolution mediums. In each case, the dissolution media were monitored spectrophotometrically to observe the appearance of brilliant blue dye in the media. The first test assessed the dissolution profile for fresh capsules of the three formulations in simulated gastric fluid (at a pH of approximately 1.3, and in the absence of any enzymes). The results of the first test are illustrated in FIG. 11. The second test assessed the dissolution profile of the three formulations in water (approximately neutral pH levels) after the formulations were stored at 40° C. and 75% relative humidity for a period of two weeks. The results of this second test are illustrated in FIG. 12. The third test assessed the dissolution profile of the three formulations in simulated gastric fluid (at a pH of approximately 1.3, and in the absence of any enzymes), after the formulations were stored at 40° C. and 75% relative humidity for a period of two weeks and four weeks. The results of this test are illustrated in FIGS. 13 and 14 for the two week storage and four week storage periods, respectively.

In reference to the results of the first test, illustrated in FIG. 11, the chart shows that little difference in dissolution profile exists for the Std Bone Gelatine, RR only, and RR RXL formulations. This result was expected as the capsules readily opened along the seam, releasing the dye, when they were fresh, immediately after they are produced.

The results of the second test, illustrated in FIG. 12, depict a difference in dissolution profiles for the three formulations after storage for two weeks. Specifically, FIG. 12 shows that the RR RXL exhibited a substantially faster and more complete dissolution profile compared to the RR only and Std Bone Gelatine formulations. The results in FIG. 12 illustrate the impact of incorporating the hydrolyzed gelatin component, which improves dissolution even though the formulations were tested in a neutral water solution. The RR Only formulation showed some improvement over the dissolution profile of the Std Bone Gelatine formulation; however, the results were not as robust as the RR RXL formulation. This is not unexpected as the dissolution medium was a neutral water solution, so the calcium carbonate was not exposed to pH levels that would cause it to effervesce and further advance dissolution of the formulation. It is important to note that the dissolution profile for the Std Bone Gelatine formulation showed a slower rate of dissolution and decreased overall dissolution, compared to the results of the first test. This is evidence that, upon storage at increased temperature (40° C.) and humidity (75% relative humidity), the dissolution properties of standard gelatin formulations is adversely affected.

The results of the third test, illustrated in FIGS. 13 and 14, depict an improved dissolution profile for the RR RXL and RR Only formulations as compared to the Std Bone Gelatine formulation. Specifically, the RR RXL and RR Only formulations exhibited a more rapid dissolution rate, as well as more complete dissolution profile, approaching 100% dissolution by the end of the time periods tested. These results illustrate the effect of incorporating calcium carbonate (in the RR RXL and RR Only formulations) and hydrolyzed gelatin (in the RR RXL formulation) on dissolution rates. It is also important to note that the dissolution profile for the Std Bone Gelatine formulation showed a slower rate of dissolution and decreased overall dissolution, compared to the results of the first test, especially in FIG. 14, illustrating dissolution after four weeks of storage. This is evidence that, upon storage at increased temperature (40° C.) and humidity (75% relative humidity), the dissolution properties of standard gelatin formulations is adversely affected.

Having described the invention in detail, it will be apparent that modifications and variations are possible. Those of skill in the art should, in light of the present disclosure, appreciate that many changes could be made in the specific embodiments that are disclosed and still obtain a like or similar result without departing from the spirit and scope of the invention, therefore all matter set forth is to be interpreted as illustrative and not in a limiting sense.

What is claimed is:

1. A rapid-release encapsulation composition comprising a water-insoluble rapid-release agent and a gelatin component.

2. The composition of claim 1, wherein the composition further comprises a gelatin hydrolysate having a molecular weight ranging from about 100 Daltons to about 2000 Daltons.

3. The composition of claim 1, wherein the water-insoluble rapid-release agent comprises a water-insoluble carbonate salt selected from bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

4. The composition of claim 2, wherein the water-insoluble rapid-release agent consists of calcium carbonate

5. The composition of claim 1, wherein the rapid-release agent is essentially insoluble at a pH ranging from about 6 to about 8, and wherein the rapid-release agent dissociates at a pH ranging from 0 to about 3.

6. The composition of claim 1, wherein the composition further comprises a mass ratio of the water-insoluble rapid-release agent to the gelatin component ranging from about 1:1 to about 1:20

7. The composition of claim 1, wherein the composition further comprises a mass ratio of the water-insoluble rapid-release agent to the gelatin component ranging from about 1:4 to about 1:9.

8. The composition of claim 1, wherein the rapid-release encapsulation composition degrades essentially completely in less than 15 minutes at a pH ranging between 0 and about 3.

9. The composition of claim 1, further comprising a plasticizer.

10. The composition of claim 9, wherein the plasticizer comprises dibutyl sebacate, diethyl phthalate, glycerine, polyethylene glycol, propylene glycol, sorbitol, erythritol, triacetin, and triethyl citrate, and mixtures thereof.

11. The composition of claim 1, wherein the gelatin component comprises a combination of a gelatin, a plasticizer, and water.

12. The composition of claim 11, wherein the gelatin component comprises from about 35% to about 60% gelatin by weight of the gelatin component, from about 15% to about 30% plasticizer by weight of the gelatin component, and from about 25% to about 40% water by weight of the gelatin component.

13. The composition of claim 11, wherein the gelatin component comprises from about 42% to about 48% gelatin by weight of the gelatin component, from about 20% to about 25% plasticizer by weight of the gelatin component, and from about 30% to about 35% water by weight of the gelatin component.

14. The composition of claim 1, wherein the gelatin component comprises a combination of a gelatin and water.

15. The composition of claim 14, therein the gelatin component comprises from about 5% to about 30% gelatin and from about 70% to about 95% water.

16. The composition of claim 14, therein the gelatin component comprises from about 10% to about 20% gelatin and from about 80% to about 90% water.

17. The composition of claim 2, wherein the composition further comprises a mass ratio of the gelatin component to the gelatin hydrolysate ranging from about 3:1 to about 99:1

18. The composition of claim 2, wherein the composition further comprises a mass ratio of the gelatin component to the gelatin hydrolysate ranging from about 4:1 to about 19:1.

19. A rapid-release encapsulation composition comprising:
a. a water-insoluble rapid-release agent;
b. a gelatin component; and
c. a gelatin hydrolysate,
wherein the composition comprises a mass ratio of the water-insoluble rapid-release agent to the gelatin component ranging from about 1:1 to about 1:20,
wherein the gelatin hydrolysate has a molecular weight ranging from about 100 Daltons to about 2000 Daltons, and
wherein the composition comprises a mass ratio of the gelatin component to the gelatin hydrolysate ranging from about 3:1 to about 99:1.

20. The composition of claim 19, wherein the mass ratio of the water-insoluble rapid-release agent to the gelatin component ranges from about 1:4 to about 1:9.

21. The composition of claim 19, wherein the plasticizer comprises dibutyl sebacate, diethyl phthalate, glycerine, polyethylene glycol, propylene glycol, sorbitol, erythritol, triacetin, and triethyl citrate, and mixtures thereof.

22. The composition of claim 19, wherein the plasticizer is selected from the group consisting of glycerine, sorbitol, erythritol, and combinations thereof.

23. The composition of claim 19, wherein the gelatin component comprises a combination of a gelatin, a plasticizer, and water.

24. The composition of claim 23, wherein the gelatin component comprises from about 35% to about 60% gelatin by weight of the gelatin component, from about 15% to about 30% plasticizer by weight of the gelatin component, and from about 25% to about 40% water by weight of the gelatin component.

25. The composition of claim 23, wherein the gelatin component comprises from about 42% to about 48% gelatin by weight of the gelatin component, from about 20% to about 25% plasticizer by weight of the gelatin component, and from about 30% to about 35% water by weight of the gelatin component.

26. The composition of claim 19, wherein the gelatin component comprises a combination of a gelatin and water.

27. The composition of claim 26, therein the gelatin component comprises from about 5% to about 30% gelatin and from about 70% to about 95% water.

28. The composition of claim 19, wherein the composition further comprises a mass ratio of the gelatin component to the gelatin hydrolysate ranging from about 4:1 to about 19:1.

29. A rapid-release encapsulation composition comprising a calcium carbonate and a gelatin component, wherein the composition has a mass ratio of the calcium carbonate to the gelatin component ranging from about 1:1 to about 1:20.

30. The rapid-release encapsulation composition of claim 29, wherein the mass ratio of the calcium carbonate to the gelatin component ranges from about 1:4 to about 1:9.

31. The composition of claim 29, wherein the composition further comprises a gelatin hydrolysate having a molecular weight ranging from about 100 Daltons to about 2000 Daltons.

32. The composition of claim 29, wherein the composition further comprises a plasticizer.

33. The composition of claim 32, wherein the plasticizer comprises dibutyl sebacate, diethyl phthalate, glycerine, polyethylene glycol, propylene glycol, sorbitol, erythritol, triacetin, and triethyl citrate, and mixtures thereof.

34. A rapid-release encapsulation composition comprising a gelatin dissolved in an aqueous solution and further comprising a water-insoluble rapid-release agent suspended in the aqueous solution.

35. The composition of claim 34, wherein the composition further comprises a gelatin hydrolysate having a molecular weight ranging from about 100 Daltons to about 2000 Daltons.

36. The composition of claim 34, wherein the aqueous solution has a pH ranging from about 6 to about 8.

37. The composition of claim 34, wherein the composition comprises gelatin and the water-insoluble carbonate salt in a combined amount comprising from about 10% to about 60% of the aqueous solution by weight.

38. The composition of claim 34, wherein the water-insoluble rapid-release agent comprises a water-insoluble carbonate salt selected from bismuth subcarbonate, calcium carbonate, cobalt carbonate, lanthanum carbonate, lead carbonate, lithium carbonate, magnesium carbonate, manganese carbonate, nickel (II) carbonate, silver carbonate, strontium carbonate, and combinations thereof.

39. The composition of claim 34, wherein the composition further comprises a plasticizer.

40. The composition of claim 39, wherein the plasticizer comprises dibutyl sebacate, diethyl phthalate, glycerine, polyethylene glycol, propylene glycol, sorbitol, erythritol, triacetin, and triethyl citrate, and mixtures thereof.

41. A chewable therapeutic composition comprising a plurality of active pharmaceutical ingredient particles, wherein each active pharmaceutical ingredient particle is encapsulated in a rapid-release coating, wherein the rapid-release coating comprises a gelatin component and a water-insoluble rapid-release agent, and wherein the rapid-release coating has a mass ratio of the water-insoluble rapid-release agent to the gelatin component ranging from about 1:1 to about 1:20.

42. The chewable therapeutic composition of claim 41, wherein the active pharmaceutical ingredient comprises an antacid.

43. A method for manufacturing a rapid-release encapsulation composition comprising the steps of:
a. dissolving a gelatin component in an aqueous medium; and
b. adding a water-insoluble rapid-release agent to the aqueous gelatin solution.

44. The method of claim 43, wherein the gelatin component of step (a) comprises a combination of a gelatin having a molecular weight ranging from about 50,000 Daltons to about 300,000 Daltons and a gelatin hydrolysate having a molecular weight ranging from about 100 Daltons to about 2000 Daltons.

45. The method of claim 43, wherein the mass ratio of the water-insoluble rapid-release agent to the gelatin component ranges from about 1:1 to about 1:20.

46. The method of claim 43, wherein step (a) further comprises the addition of a plasticizer.

47. The method of claim 46, wherein the plasticizer comprises dibutyl sebacate, diethyl phthalate, glycerine, polyethylene glycol, propylene glycol, sorbitol, erythritol, triacetin, and triethyl citrate, and mixtures thereof.

48. The method of claim 43, wherein the gelatin component comprises a combination of a gelatin and a plasticizer.

49. The method of claim 44, wherein the mass ratio of the gelatin having a molecular weight ranging from about 50,000 Daltons to about 300,000 Daltons to the gelatin hydrolysate having a molecular weight ranging from about 100 Daltons to about 2000 Daltons ranges from about 3:1 to about 99:1.

50. The method of claim 43, wherein step (a) comprises dissolving about 5% to about 25% of the gelatin component by weight of the combined aqueous gelatin solution in about 75% to about 95% of the aqueous medium by weight of the combined aqueous gelatin solution.

51. The method of claim 44, wherein step (a) comprises dissolving about 10% to about 20% by weight of the gelatin having a molecular weight ranging from about 50,000 Daltons to about 300,000 Daltons and about 1% to about 5% by weight of the gelatin hydrolysate having a molecular weight ranging from about 100 Daltons to about 2000 Daltons in about 70% to about 90% aqueous medium by weight of the combined aqueous gelatin solution.

52. The method of claim 43, wherein step (a) comprises dissolving about 35% to about 60% gelatin by weight of the gelatin component, about 15% to about 30% of a plasticizer by weight of the gelatin component, and about 25% to about 40% aqueous medium by weight of the gelatin component.

53. The method of claim 43, wherein step (a) comprises dissolving about 42% to about 48% gelatin by weight of the gelatin component, about 20% to about 25% of a plasticizer by weight of the gelatin component, and about 30% to about 35% aqueous medium by weight of the gelatin component.

54. The method of claim 43, wherein step (a) comprises dissolving from about 32% to about 40% by weight of a gelatin having a molecular weight ranging from about 50,000 Daltons to about 300,000 Daltons, from about 17% to about 22% by weight plasticizer, from about 26% to about 31% by weight of the aqueous medium, and from about 2% to about 6% by weight of a gelatin hydrolysate having a molecular weight ranging from about 100 Daltons to about 2000 Daltons, and wherein step (b) comprises adding about 10% to about 15% by weight calcium carbonate.

55. The method of claim 43, wherein the aqueous medium comprises water.
